(19) United States
(12) Patent Application Publication
(10) Pub. No.: US 2004/0131706 A1
(43) Pub. Date: Jul. 8, 2004

(54) Pharmaceutical preparation

(76) Inventors: Reiner Rittinghausen, Inning/Bachern (DE); Volkmar Koch, Utting/Ammersee (DE)

Correspondence Address: COHEN, PONTANI, LIEBERMAN & PAVANE, 551 FIFTH AVENUE, SUITE 1210, NEW YORK, NY, 10176, US

(21) Appl. No.: 10/477,483

(22) PCT Filed: May 29, 2002
(86) PCT No.: PCT/EP02/05920

(30) Foreign Application Priority Data
May 30, 2001 (DE) 201 09 044.9

(51) Int. Cl. A61K 35/78

(52) U.S. Cl. 424/725

(57) ABSTRACT

The invention relates to the use of a pharmaceutical preparation for producing a medicament used for treating allergic skin diseases, atopic eczema, skin diseases caused by fungi, urticaria, neurodermatitis, psoriasis, psora, skin eczemas, pruritus of the skin, inflammatory skin diseases and/or viral diseases, including chickenpox, varicellas, herpes and herpes zoster. The inventive pharmaceutical preparation comprises natural and/or synthetic active ingredient(s) extracted from the plants Centella asiatica, Mahonia aquifolium, Viola tricolor and/or from plant parts thereof.

The invention concerns a pharmaceutical preparation and its use in the treatment of allergic skin diseases, atopic dermatitis, skin diseases caused by fungi, urticaria, neurodermatitis, psoriasis, eczema, pruritus, inflammatory skin diseases, and/or viral diseases, including chickenpox, varicellas, herpes, and herpes zoster.

Plant extracts from Centella asiatica, Mahonia aquifolium, Viola tricolor, and/or their plant parts for the treatment of viral diseases, especially viral skin diseases or virus-induced skin changes, including chickenpox, varicellas, herpes, rubella, and herpes zoster, are not known from the state of the art.

Eczemas are erythematous and scaly, sometimes oozing, skin changes. Typical sites of appearance are the elbow, popliteal region, neck, throat, and face. Patients with pruritus characteristically have dry skin.

Pruritus is an alarm signal of the skin indicating an altered external or internal situation. The itching sensation is caused by various mechanical, physical, and chemical effects. Emotional factors also play a role.

Pruritus is triggered principally by four mechanisms, which are partly interdependent or may mutually affect one another,

1. Release of pruritus-triggering tissue substances, for example, histamine, from the defense cells of the skin, for example, from the mast cells;

2. Release of proteolytic enzymes that trigger pruritus and cause inflammation, for example, from the phagocytes;

3. Direct effect on neural receptors and thus secretion of neural hormones, such as neuropeptides; and

4. Stimulation of pruritus in the brain.

This type of stimulus in or on the skin triggers a so-called scratch reflex. Since inflammation of the skin caused by the resulting irritation of nerve endings can trigger further itching, the result can be a vicious circle of scratching, renewed itching, even stronger scratching, and so on.

Neurodermatitis is one of the most common skin diseases that trigger itching and scratching. Its clinical picture is characterized by a variable eczema of highly varying appearance.

A preparation for the treatment of pruritic eczemas is commercially available. It contains 1 wt. % balsam of Peru (balsamum peruvianum) and 5 wt. % zinc oxide as active ingredients. Other ingredients are extracts of Centella asiatica, Mahonia aquifolium, Viola tricolor, acidum arsenicosum, Calendula officinalis, Lytta vesicatoria, and Semecarpus anacardium. However, this preparation has disadvantages.

Balsam of Peru is an active substance that has long been known and is used, for example, in drugs, mainly externally, as a vulnerary, an ingredient of antihemorrhoid and antiscabies preparations, and in liniments. Balsam of Peru or balsamum peruvianum contains at least 45%, and at most 75%, of ether-extractable cinnamic acid and benzoic acid benzyl esters, 25-30% resins, benzoic acid, cinnamic acid, vanillin, nerolidol, and farnesol. However, in addition to its antiseptic action, balsam of Peru produces undesired side effects in the form of contact allergies relatively frequently.

Another disadvantage of the above preparation for the treatment of pruritic eczema is its high content of zinc oxide, which, as an ingredient of ointments and pastes, causes drying of the skin, and thus an unpleasant skin sensation.

It would be very desirable to have available a pharmaceutical preparation for the treatment of pruritic eczemas, such as neurodermatitis, which avoids the disadvantages described above. In particular, it would be desirable to have available a pharmaceutical preparation that produces a pleasant skin sensation for the patient and has no side effects in the form of allergic skin reactions.

Furthermore, it would be very desirable to make available a pharmaceutical preparation that is also suitable for the treatment of viral diseases, especially the treatment of viral skin diseases and/or virus-induced skin changes, including chickenpox, varicellas, herpes, rubella, and herpes zoster.

Therefore, the objective of the present invention is to make available a pharmaceutical preparation that overcomes the disadvantages of the state of the art and can be used for the treatment of eczemas and pruritic eczemas, especially pruritus, such as the pruritus that occurs in neurodermatitis.

A further objective of the invention is to make available a pharmaceutical preparation that is suitable for the treatment of viral diseases, especially the treatment of viral skin diseases and/or virus-induced skin changes, including chickenpox, varicellas, herpes, rubella, and herpes zoster.

Yet another objective of the present invention is to make available a pharmaceutical preparation that is suitable for the treatment of allergic skin diseases, atopic eczema, fungal skin diseases, urticaria, neurodermatitis, psoriasis, and/or inflammatory skin diseases.

Further details are given in the following description of the invention.

In accordance with the invention, it is proposed that a pharmaceutical preparation be used to produce a drug for the treatment of allergic skin diseases, atopic eczema, fungal skin diseases, urticaria, neurodermatitis, psoriasis, eczema, pruritus, inflammatory skin diseases, and/or viral diseases, including chickenpox, varicellas, herpes, and herpes zoster, such that the pharmaceutical preparation contains natural and/or synthetic active ingredients from the plants Centella asiatica, Mahonia aquifolium, Viola tricolor, and/or their plant parts.

The use, in accordance With the invention, of the pharmaceutical preparation for the treatment of viral diseases, especially the treatment of viral skin diseases or virus-induced skin changes, is advantageous.

In addition, in accordance with the invention, a pharmaceutical preparation is prepared, which contains natural and/or synthetic active substance(s) from the plants Centella asiatica, Mahonia aquifolium, Viola tricolor, and/or their plant parts, and from which a content of 5 wt. % zinc oxide and/or a content of 1% balsam of Peru, based on the total composition, is excluded.

Advantageous uses and refinements of the pharmaceutical preparation of the invention are defined in the dependent claims.

Unless otherwise indicated, all values in wt. % are based on the total composition of the pharmaceutical preparation.

The active ingredients of the preparation of the invention are preferably obtained as extracts from the plants Centella asiatica, Mahonia aquifolium, Viola tricolor, and/or their plant parts and possibly from the other plants, plant parts, or animals described below. Especially preferred is the preparation of a primary tincture that contains natural and/or synthetic active ingredients from the plants Centella asiatica, Mahonia aquifolium, Viola tricolor, and/or their plant parts, and possibly from the other plants or plant parts described below.

Within the context of the present invention, extracts are understood to be concentrated preparations from active substances, which are possibly adjusted to a certain concentration of active substance.

Alcoholic extracts may be used directly as liquid extracts or as dry extracts after removal of the menstruum.

Alcoholic extracts can usually be used, for example, with an ethanol content of ≧30 wt. % and a water content of ≦70 wt. %, based on the total weight of the extractant. The alcoholic extracts preferably contain ≧42 wt. %, ≧62 wt. %, ≧86 wt. %, or ≧94 wt. % ethanol, with the remainder consisting of water.

The ethanol/water mixtures described above are preferably used as extractants for extraction from the three plants, also called drugs, Centella asiatica, Mahonia aquifolium, Viola tricolor, and/or their plant parts.

Centella asiatica can be extracted, for example, according to the specifications 4a HAB (Homeopathic Pharmacopeia 2001) with 62 wt. % ethanol and 38 wt. % water. Preferably the dried, above-ground parts of Centella asiatica (Linné) Urban are used for the extraction.

Mahonia aquifolium can be extracted, for example, according to the specifications 4a HAB (Homeopathic Pharmacopeia 2001) with 62 wt. % ethanol and 38 wt. % water. Preferably the branch and twig bark and the dried twig tips of Mahonia aquifolium (Pursh) are used for the extraction.

Viola tricolor can be extracted, for example, according to the specifications 2a HAB (Homeopathic Pharmacopeia 2001) with 86 wt. % ethanol and 14 wt. % water. Preferably the fresh, above-ground parts of Viola tricolor are used for the extraction.

Other ethanol/water mixtures may also be used for the extraction from these three drugs, Centella asiatica, Mahonia aquifolium, and Viola tricolor.

More lipophilic extracts can be produced with suitable extractants, such as C₂-C₈ alkyl compounds, e.g., pentane or n-hexane, or with compressed ethylene, ethane, or carbon dioxide under supercritical conditions.

WO 00/12,107 describes processes for extracting plants or plant parts with carbon dioxide, ethylene, and ethane under supercritical conditions, which are fully incorporated in the present application by reference. Experts in this field are already familiar with extraction with subcritical and supercritical carbon dioxide.

Furthermore, Hagers Handbuch [Hager's Handbook], Vol. 2, Springer Verlag 1991, pages 1,024-1,031, describes processes for producing plant or drug extracts, which are likewise fully incorporated in the present application by reference.

After removal of the menstruum or extractant, dry or inspissated extracts are obtained. Dry extracts can be processed into powders. In the case of inspissation, the extracts are viscous, often with a water content of ≦5 wt. %, based on the inspissated extract.

In the context of the present invention, a primary tincture is understood to mean mixtures of juices expressed from plants and/or plant extracts with solvents, such as ethanol, water, and/or glycerol, or extracts of plants or animals, their secretions or their parts. Primary tinctures are preferably prepared with liquid drug vehicles, such as ethanol of various concentrations, water, and/or glycerol.

In the context of the present invention, synthetic active substances are understood to mean active substances that are identical with or similar to natural substances and that were prepared by entirely synthetic or partly synthetic means.

Surprisingly, it was found that a pharmaceutical preparation of the invention, which contains less than 1 wt. % balsam of Peru, produces no side effects at all, e.g., in the form of allergic skin reactions. At the same time, however, the outstanding antipruritic effect of the preparation was assured. This prevents the scratch reflex from being triggered by itching and then leading to more intense itching. In one embodiment of the present invention, the preparation thus contains <1 wt. %, preferably ≦0.5 wt. %, more preferably ≦0.1 wt. % and most preferably ≦0.01 wt. % of balsam of Peru.

In a preferred embodiment, the pharmaceutical preparation contains no balsam of Peru. In this way, allergic skin reactions are avoided, e.g., in repeated treatment or long-term therapy of pruritus and eczema, including especially in very sensitive patients.

The efficacy of the pharmaceutical preparation against fungi and viruses is also surprising, especially in the treatment of viral and/or fungal skin diseases and/or skin changes induced by viruses or fungi.

In another preferred embodiment of the present invention, the pharmaceutical preparation also contains <5 wt. %, preferably ≦3 wt. %, more preferably ≦1 wt. %, even more preferably ≦0.1 wt. %, and most preferably no zinc oxide. Preferred lower limits for the zinc oxide concentration of the pharmaceutical preparation of the invention are 0.5 wt. %, and especially 0.1 wt. %. Most preferably, the pharmaceutical preparation of the invention contains no zinc oxide. The low zinc oxide concentration of the pharmaceutical preparation of the invention prevents drying of the skin, so that treatment with the preparation of the invention produces a pleasant sensation in the skin.

The preparation of the invention preferably contains active ingredients extracted from the plants Centella asiatica, Mahonia aquifolium, Viola tricolor, and/or their plant parts and possibly from the other plants or plant parts described below.

The preparation of the invention may also be present in the form of a primary tincture or tinctures obtained from the plants Centella asiatica, Mahonia aquifolium, Viola tricolor, and/or their plant parts, and possibly from the other plants or plant parts described below.

In a preferred embodiment of the present invention, the pharmaceutical preparation contains as one of its ingredients an extract, especially a primary tincture, from the plant Centella asiatica and/or from its plant parts in a concentration of 1 wt. % to 10 wt. %, preferably 3 wt. % to 7 wt. %, more preferably 4 wt. % to 6 wt. %, and most preferably 5 wt.%.

Centella asiatica (Asiatic pennywort) contains about 0.1 wt. % of essential oils, flavonol derivatives, and ursane derivatives, especially (free) asiaticoside and its trisaccharide ester. Active substances from Centella asiatica are efficient in skin diseases with lichenification and pruritus and can be used in the form of ointments and tinctures to promote wound healing and especially to promote the antimicrobial and antiphlogistic effect.

In a preferred embodiment, the pharmaceutical preparation of the invention also contains an extract, especially a primary tincture, from the plant Mahonia aquifolium in a concentration of 1 wt. % to 10 wt. %, preferably 3 wt. % to 7 wt. %, more preferably 4 wt. % to 6 wt. %, and most preferably 5 wt. %.

Mahonia aquifolium contains the alkaloids berberine, oxyacanthine, and bermamine as constituents of its trunk and root bark. Active substances from Mahonia aquifolium (Berberis aquifolium) can be advantageously used in the treatment of dry skin rashes.

In a preferred embodiment, the pharmaceutical preparation of the invention also contains an extract, especially a primary tincture, from the plant Viola tricolor in a concentration of 1 wt. % to 10 wt. %, preferably 3 wt. % to 7 wt. %, more preferably 4 wt. % to 6 wt. %, and most preferably 5 wt. %. Viola tricolor (pansy) contains the constituents: flavonoids, traces of essential oils, the glycoside violutoside (violutin), in which methyl salicylate is bound with the disaccharide vicianose, as well as tannin, mucilage, and sugar. Viola tricolor or its active constituents are effective for the treatment of chronic skin diseases and rashes, especially in pediatrics (crusta lactea), and including especially the treatment of eczema.

The pharmaceutical preparation of the invention also preferably contains coloring agents. Examples of preferred coloring agents are curcumin, riboflavin, and/or β-carotene.

The pharmaceutical preparation of the invention may be present in the form of an ointment, cream, gel, tablet, or solution. However, in accordance with the invention, the pharmaceutical preparation is preferably formulated as an ointment, more preferably as a hydrophilic ointment, and especially as a water-containing hydrophilic ointment.

Creams can also be advantageously used in accordance with the present invention.

The ointment base is preferably selected on the basis of the fact that water-soluble drugs can be better absorbed by the skin from hydrophobic ointment bases, whereas fat-soluble drugs can be absorbed by the skin more rapidly from hydrophilic ointment bases. Accordingly, the pharmaceutical preparation of the invention preferably contains DAB or DAC bases or constituents as the ointment or cream base, including especially hydroxyethyl cellulose and/or wool grease alcohol ointment (DAB: German Pharmacopeia 2000/2001, DAC: German Drug Codex 2000/2001).

Hydrophilic gel preparations, hydrophilic ointments, and hydrophilic creams may contain zinc oxide, since these types of preparations have a cooling effect and deliver moisture to the skin, so that dry skin is avoided. Pruritus is also reduced by the moisture-delivering property of the aforementioned hydrophilic formulations.

In another preferred embodiment of the present invention, the preparation also contains natural and/or synthetic active ingredients, especially in the form of a primary tincture, from the plants Calendula officinalis and/or Semecarpus anacardium and/or their plant parts.

The constituents of Calendula officinalis (marigold) include essential oils, calendulin, calendulosides, such as saponins and oleanolic acid glycosides, triterpenes, β-carotene, lycopene, xanthophylls, and flavonoids. The active substances present in Calendula officinalis have anti-inflammatory, bactericidal, and granulation-promoting effects, especially in ointments used to treat poorly healing wounds and ulcers.

The constituents of Semecarpus anacardium (Malacca kidney bean) include phenolic compounds, such as cardol, tannin, resin, and pigment, and the seeds contain about 47% fatty oil with anacardic acid. Semecarpus anacardium or its active substances are effective as rubefacients and in the treatment of warts and corns.

The preparation of the invention may also contain arsenous acid (arsenic(III) oxide) as an active ingredient.

In addition, the preparation may contain natural and/or synthetic active substances from Lytta vesicatoria. Lytta vesicatoria is a beetle, which contains about 0.5 to 1% cantharidin, resin, fat, and pigment. Preparations from Lytta vesicatoria are prescribed, for example, for the treatment of burns.

Furthermore, the pharmaceutical preparation of the invention may contain active ingredients selected from the vitamin group, especially vitamin E, β-carotene, biotin, and coenzyme Q₁₀. Other advantageous ingredients are curcumin and curcumin rhizome extract.

In an especially preferred embodiment, the preparation contains active ingredients selected from the group comprising essential oils, flavonol derivatives, ursane derivatives, berberin, oxyacanthine, bermamine, violutoside (violutin), tannins, sugars, mucilages, calendulin, calendulosides, triterpenes, β-carotene, lycopene, xanthophylls, anacardic acid, arsenic(III) oxide, cantharidin, vitamins, especially vitamin E, β-carotene, biotin, coenzyme Q₁₀, zinc oxide, ether-extractable cinnamic acid and benzoic acid benzyl esters, resins, benzoic acid, cinnamic acid, curcumin, curcumin rhizome extract, vanillin, nerolidol, and/or farnesol.

In addition, it is preferred that the pharmaceutical preparation of the invention be ready to use. In the context of the present invention, “ready to use” is understood to mean that the pharmaceutical preparation is present in the form of a ready-to-use ointment that can be applied to the itching, inflamed, or eczematous areas of the skin.

Another object of the present invention is a tube that contains a pharmaceutical preparation of the invention.

The present invention also concerns a method of preparing a pharmaceutical preparation, which comprises the mixing of extracts, especially the preparation of primary tinctures, from Centella asiatica, Mahonia aquifolium, and Viola tricolor, from which a content of 5 wt. % zinc oxide and/or a content of 1% balsam of Peru, based on the total composition, is excluded.

In this method of the invention for preparing a pharmaceutical preparation for producing a drug for the treatment of neurodermatitis, psoriasis, chickenpox, varicellas, herpes, eczema, pruritus, and/or inflammatory skin diseases, it is preferred that the weight ranges specified above for the pharmaceutical preparation, especially the primary tinctures, from Centella asiatica, Mahonia aquifolium, and/or Viola tricolor, be observed.

Furthermore, in the method of the invention, it is preferred that no balsam of Peru and/or less than 5 wt. %, preferably less than 3 wt. %, more preferably less than 1 wt. %, and most preferably no zinc oxide be added.

In another preferred embodiment of the method of the invention, the preparation is prepared as an ointment or cream, and DAB or DAC bases of constituents are preferably used as the ointment or cream base, such as especially hydroxyethyl cellulose and/or wool grease alcohol ointment. Preferably, a hydrophilic ointment or cream, especially a water-containing hydrophilic ointment or cream, is used. Furthermore, in the method of the invention, active ingredients selected from the vitamin group, preferably vitamin E, β-carotene, biotin, and coenzyme Q₁₀, and other active substances specified above are added.

Another aspect of the present invention concerns the use of a pharmaceutical preparation of the invention prepared as described above, which contains natural and/or synthetic active substances from the plants Centella asiatica, Mahonia aquifolium, and Viola tricolor, from which a content of 5 wt. % zinc oxide and/or a content of 1 wt. % balsam of Peru, based on the total composition, is excluded, for the production of a drug for the treatment of neurodermatitis, psoriasis, chickenpox, varicellas, herpes, eczema, and/or pruritus, especially the treatment of neurodermatitis.

The following examples illustrate the present invention.

### EXAMPLE 1

### Formulation for a Vegetable Primary Tincture for External Use in Pruritic Eczema

| Constituents | Wt. % | Monographs |
|---|---|---|
| arsenous acid dil. D4 | 1 | Inflammations in all tissues |
| Calendula officinalis | 2 | Poorly healing wounds, |
| | | skin suppuration |
| Centella asiatica | 5 | Skin diseases with |
| | | lichenification and pruritus |
| Lytta vesicatoria | 1 | Acute inflammation of the |
| (= Cantharis) | | skin with blistering |
| dil. D4 | | |
| Mahonia aquifolium | 1 | Dry skin rash |
| balsam of Peru | 0 | |
| Semecarpus | 1 | Skin rashes |
| anacardium | | |
| Viola tricolor | 3 | Eczema |
| zinc oxide | 0 | |

### EXAMPLE 2

### Formulation for a Vegetable Primary Tincture for External Use in Pruritic Eczema

| Components | Wt. % | Monographs |
|---|---|---|
| Centella asiatica | 5 | Skin diseases with |
| | | lichenification and pruritus |
| Mahonia aquifolium | 5 | Dry skin rashes |
| Viola tricolor | 5 | Eczema |

Even after the repeated use of an ointment or cream based on the formulations described above, no contraindications or side effects were observed. With both formulations, an outstanding antipruritic effect of the pharmaceutical preparation was assured.

The formulations specified in Example 1 and Example 2 can be used in accordance with the invention for the treatment of allergic skin diseases, atopic eczema, fungal skin diseases, urticaria, neurodermatitis, psoriasis, eczema, pruritus, inflammatory skin diseases, and/or viral diseases, including chickenpox, varicellas, herpes, and herpes zoster.

1. Use of a pharmaceutical preparation to produce a drug for the treatment of allergic skin diseases, atopic eczema, fungal skin diseases, urticaria, neurodermatitis, psoriasis, eczema, pruritus, inflammatory skin diseases, and/or viral diseases, including chickenpox, varicellas, herpes, and herpes zoster, such that the pharmaceutical preparation contains natural and/or synthetic active substance(s) from the plants Centella asiatica, Mahonia aquifolium, Viola tricolor, and/or their plant parts.

2. Use of the preparation in accordance with claim 1, characterized by the fact that the preparation is an alcoholic extract from the plants Centella asiatica, Mahonia aquifolium, Viola tricolor, and/or their plant parts.

3. Use of the preparation in accordance with claim 1 or claim 2, characterized by the fact that the preparation is a lipophilic extract from the plants Centella asiatica, Mahonia aquifolium, Viola tricolor, and/or their plant parts, preferably an extract produced by means of compressed ethylene, ethane, or carbon dioxide under supercritical conditions, or an alkyl extract, preferably a C₂-C₈ alkyl extract, especially a carbon dioxide extract or hexane extract.

4. Use of the preparation in accordance with any of the preceding claims, characterized by the fact that the preparation contains <1 wt. %, preferably ≦0.5 wt. %, more preferably ≦0.1 wt. %, and most preferably ≦0.01 wt. % of balsam of Peru, based on the total weight of the composition.

5. Use of the preparation in accordance with any of the preceding claims, characterized by the fact that the preparation contains <5 wt. %, preferably ≦3 wt. %, more preferably ≦1 wt. %, and most preferably ≦0.1 wt. % zinc oxide, based on the total weight of the composition.

6. Use of the preparation in accordance with any of the preceding claims, characterized by the fact that the preparation contains no balsam of Peru and/or zinc oxide.

7. Use of the preparation in accordance with any of the preceding claims, characterized by the fact that the preparation contains a primary tincture with active substance(s) from the plants Centella asiatica, Mahonia aquifolium, Viola tricolor, and/or their plant parts.

8. Use of the preparation in accordance with any of the preceding claims, characterized by the fact that the preparation contains active substances, preferably in the form of a primary tincture, from the plant Centella asiatica and/or its plant parts in a concentration of 1 wt. % to 10 wt. %, preferably 3 wt. % to 7 wt. %, more preferably 4 wt. % to 6 wt. %, and most preferably 5 wt. %, based on the total weight of the preparation.

9. Use of the preparation in accordance with any of the preceding claims, characterized by the fact that the preparation contains active substances, preferably in the form of a primary tincture, from the plant Mahonia aquifolium and/or its plant parts in a concentration of 1 wt. % to 10 wt. %, preferably 3 wt. % to 7 wt. %, more preferably 4 wt. % to 6 wt. %, and most preferably 5 wt. %, based on the total weight of the preparation.

10. Use of the preparation in accordance with any of the preceding claims, characterized by the fact that the preparation contains active substances, preferably in the form of a primary tincture, from the plant Viola tricolor and/or its plant parts in a concentration of 1 wt. % to 10 wt. %, preferably 3 wt. % to 7 wt. %, more preferably 4 wt. % to 6 wt. %, and most preferably 5 wt. %, based on the total weight of the preparation.

11. Use of the preparation in accordance with any of the preceding claims, characterized by the fact that the preparation additionally contains natural and/or synthetic coloring agents, selected especially from the group comprising curcumin, riboflavin, and βcarotene.

12. Use of the preparation in accordance with any of the preceding claims, characterized by the fact that the preparation is present in the form of an ointment, cream, gel, tablet, or solution, and a hydrophilic ointment, especially a water-containing hydrophilic ointment, is preferred.

13. Use of the preparation in accordance with any of the preceding claims, characterized by the fact that the preparation contains hydroxyethyl cellulose and/or wool grease alcohol as the ointment or cream base.

14. Use of the preparation in accordance with any of the preceding claims, characterized by the fact that the preparation additionally contains natural and/or synthetic active substance(s), especially in the form of a primary tincture from the plants Calendula officinalis and/or Semecarpus anacardium and/or their plant parts.

15. Use of the preparation in accordance with any of the preceding claims, characterized by the fact that the preparation additionally contains arsenous acid as an active substance.

16. Use of the preparation in accordance with any of the preceding claims, characterized by the fact that the preparation additionally contains natural and/or synthetic active substance(s) from Lytta vesicatoria.

17. Use of the preparation in accordance with any of the preceding claims, characterized by the fact that the preparation additionally contains active substances selected from the vitamin group, preferably vitamin E, β-carotene, and coenzyme Q₁₀.

18. Use of the preparation in accordance with any of the preceding claims, characterized by the fact that the preparation additionally contains active substances selected from the group comprising curcumin and curcumin rhizome extract.

19. Use of the preparation in accordance with any of the preceding claims, characterized by the fact that the preparation is ready to use.

20. Pharmaceutical preparation, characterized by the fact that it has a composition in accordance with any of the preceding claims 1 to 17, from which a content of 5 wt. % zinc oxide and/or a content of 1 wt. % balsam of Peru, based on the total composition, is excluded.

21. Tube containing a pharmaceutical preparation in accordance with any of the preceding claims.
